(19) United States
(12) Patent Application Publication
(10) Pub. No.: US 2015/0164890 A1
(43) Pub. Date: Jun. 18, 2015

(54) PHARMACEUTICAL COMPOSITIONS COMPRISING CRYSTALLINE POSACONAZOLE

(71) Applicant: SANDOZ AG, Basel (CH)

(72) Inventors: Susanne Palmberger (née: Bartsch), Kundl/Tyrol (AT); Christine Mohr, Kundl/Tyrol (AT); Gottfried Stubauer, Kundl/Tyrol (AT)

(21) Appl. No.: 14/407,336

(22) PCT Filed: Jun. 13, 2013
(86) PCT No.: PCT/EP2013/062298

§ 371 Date: Dec. 11, 2014

(30) Foreign Application Priority Data
Jun. 14, 2012 (EP) 12172032.0

(51) Int. Cl. A61K 31/496

(52) U.S. Cl. CPC A61K 31/496

(57) ABSTRACT

The present invention relates to a pharmaceutical composition which comprises crystalline posaconazole and one or more non-ionic surfactants, wherein at least one non-ionic surfactant is an ethoxylated hydrogenated castor oil.

The present invention relates to a pharmaceutical composition which comprises crystalline posaconazole and one or more non-ionic surfactants, wherein at least one non-ionic surfactant is an ethoxylated hydrogenated castor oil. Preferably, at least 90 weight-% of the posaconazole comprised in the pharmaceutical composition are present as crystalline form IV. Further, the present invention relates to a process for the preparation of said pharmaceutical composition, said pharmaceutical composition for use in a method of treating or preventing fungal infections in mammals in need of such treating or preventing such infections, and to the use of a combination of posaconazole, preferably of crystalline form IV, and at least one ethoxylated hydrogenated castor oil for improving the long-term stability of liquid dosage forms comprising posaconazole.

### BACKGROUND PRIOR ART

Posaconazole (CAS Registry Number 171228-49-2; CAS Name: 2,5-anhydro-1,3,4-trideoxy-2-C-(2,4-difluorophenyl)-4-[[4-[4-[4-[1-[(1S,2S)-1-ethyl-2-hydroxypropyl]-1,5-dihydro-5-oxo-4H-1,2,4-triazol-4-yl]phenyl]-1-piperazinyl]phenoxy]methyl]-1-(1H-1,2,4-triazol-1-yl)-D-threo-pentitol) which is represented by the following general formula (I)

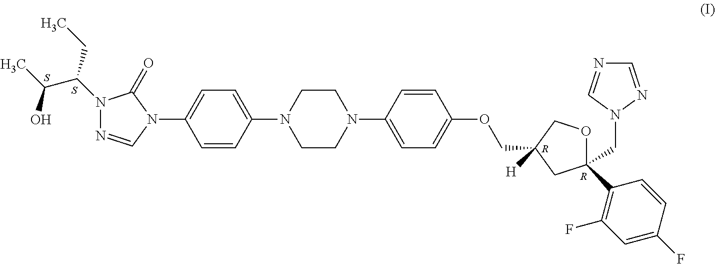

is known as an antifungal agent. It is available as an oral suspension (40 mg/mL) under the trademark NOXAFIL® from Schering Corporation, Kenilworth, N.J.

In WO 2010/000668 A1, the crystalline form IV of posaconazole is disclosed, and it is discussed that the use of this crystalline form IV for the preparation of liquid suspensions or dispersions allows for avoiding time consuming and costly micronization techniques which are generally applied for treating known crystalline form I of posaconazole. This is described to be due to the fact that crystalline form IV of posaconazole has a smaller median particle size and a larger specific surface area when compared to known non-micronized crystalline form I of posaconazole. As far as the pharmaceutical compositions are concerned, WO 2010/000668 A1 describes the use of thickening agents and non-ionic surfactants. As to conceivable respective compounds, reference is made to WO 02/080678 A1. The specific examples of WO 2010/000668 A1 illustrating the advantageous characteristics of the crystalline form IV of posaconazole show compositions comprising, as sole non-ionic surfactant, Polysorbate 80.

WO 02/080678 A1, referred to in the above-discussed WO 2010/000668 A1, discloses liquid suspensions comprising an antifungally effective amount of posaconazole, at least one thickening agent, at least one non-ionic surfactant, and a pharmaceutically effective carrier. Concerning the non-ionic surfactant, a large number of different compounds are described, such as block copolymers of ethylene oxide and propylene oxide, glycol or glyceryl esters of saturated or unsaturated C₈ to C₂₀ acids, preferably, polyoxyethylene esters of saturated or unsaturated C₈ to C₂₀ acids, polyoxyethylene ethers of saturated or unsaturated C₈ to C₂₀ acids, and polyvinyl alcohols or sorbitan esters of saturated or unsaturated C₁₀ to C₂₀ acids. As suitable polyoxyethylene esters of fatty acids, both polyoxyethylene castor oil and hydrogenated castor oil derivatives are mentioned. Preferred non-ionic surfactants according to WO 02/080678 A1 are sorbitan esters of a saturated or unsaturated C₁₀ to C₂₀ acid, and fatty acid esters of sorbitan selected from sorbitan monolaurate, sorbitan monooleate, sorbitan sesquioleate, sorbitan trioleate, sorbitan monopalmitate, sorbitan monostearate and sorbitan tristearate, or mixtures thereof are disclosed as being especially preferred. Consequently in the examples of WO 02/080678 A1, pharmaceutical compositions are disclosed which contain the sorbitan ester Polysorbate 80 as sole non-ionic surfactant.

Generally, for pharmaceutical compositions, there is the need for a long-term stability. In particular for liquid suspensions containing the pharmaceutically active compound suspended in a liquid medium, it is desired that the particle size of the pharmaceutically active compound does not, or not significantly, change over time in order to avoid sedimentation effects. In particular for liquid suspensions containing the pharmaceutically active compound exhibiting a comparatively small particle size, this long-term stability with respect to the particle size is a challenge.

Therefore, it was an object of the present invention to provide pharmaceutical compositions comprising posaconazol, which pharmaceutical compositions have advantageous characteristics regarding the long-term stability with respect to the particle size.

### SUMMARY OF THE INVENTION

Surprisingly, it was found that this object can be solved by providing pharmaceutical compositions comprising crystalline posaconazole and at least one non-ionic surfactant wherein at least one non-ionic surfactant is an ethoxylated hydrogenated castor oil. In particular, it was found that this object can be solved by providing pharmaceutical compositions comprising crystalline posaconazole and at least one non-ionic surfactant wherein at least one non-ionic surfactant is an ethoxylated hydrogenated castor oil and wherein preferably at least 90 weight-%, preferably at least 95 weight-%, more preferably at least 98 weight-% of the posaconazole comprised in the pharmaceutical compositions is present as posaconazole of crystalline form IV.

The present invention relates to a pharmaceutical composition, comprising crystalline posaconazole and one or more non-ionic surfactants, wherein at least one non-ionic surfactant is an ethoxylated hydrogenated castor oil. According to a preferred embodiment, the present invention relates to a pharmaceutical composition, comprising crystalline posaconazole and one or more non-ionic surfactants, wherein at least one non-ionic surfactant is an ethoxylated hydrogenated castor oil, wherein at least 90 weight-%, preferably at least 95 weight-%, more preferably at least 98 weight-% of the posaconazole comprised in the pharmaceutical composition are present as crystalline form IV, having an X-ray powder diffraction pattern comprising peaks at 2-theta angles of about 3.2°±0.2°, 6.6°±0.2°, 10.9°±0.2°, 16.9°±0.2°, 18.4°±0.2° and 25.1°±0.2°, measured with Cu—K alpha_{1,2} radiation, and/or having an attenuated total reflectance infrared spectrum comprising absorption bands at wavenumbers of about 3647 cm⁻¹±2 cm⁻¹, 3472 cm⁻¹±2 cm⁻¹, 2867 cm⁻¹±2 cm⁻¹, 1687 cm⁻¹±2 cm⁻¹, 1512 cm⁻¹±2 cm⁻¹, 1230 cm⁻¹±2 cm⁻¹, 1136 cm⁻¹±2 cm⁻¹, 916 cm⁻¹±2 cm⁻¹, 853 cm⁻¹±2 cm⁻¹, 819 cm⁻¹±2 cm⁻¹ and 681 cm⁻¹±2 cm⁻¹.

Further, the present invention relates to a process for the preparation of a pharmaceutical composition, preferably of the pharmaceutical composition described above, the process comprising
(aa) providing crystalline posaconazole, wherein preferably at least 90 weight-%, more preferably at least 95 weight-%, more preferably at least 98 weight-% of the posaconazole are present as crystalline form IV having an X-ray powder diffraction pattern comprising peaks at 2-theta angles of about 3.2°±0.2°, 6.6°±0.2°, 10.9°±0.2°, 16.9°±0.2°, 18.4°±0.2° and 25.1°±0.2°, measured with Cu—K alpha_{1,2} radiation, and/or having an attenuated total reflectance infrared spectrum comprising absorption bands at wavenumbers of about 3647 cm⁻¹±2 cm⁻¹, 3472 cm⁻¹±2 cm⁻¹, 2867 cm⁻¹±2 cm⁻¹, 1687 cm⁻¹±2 cm⁻¹, 1512 cm⁻¹±2 cm⁻¹, 1230 cm⁻¹±2 cm⁻¹, 1136 cm⁻¹±2 cm⁻¹, 916 cm⁻¹±2 cm⁻¹, 853 cm⁻¹±2 cm⁻¹, 819 cm⁻¹±2 cm⁻¹ and 681 cm⁻¹±2 cm⁻¹;
(bb) mixing one or more non-ionic surfactants with the posaconazole provided in (aa), wherein at least one non-ionic surfactant is an ethoxylated hydrogenated castor oil, the at least one ethoxylated hydrogenated castor oil preferably being selected from the group consisting of PEG-5 hydrogenated castor oil; PEG-7 hydrogenated castor oil; PEG-16 hydrogenated castor oil; PEG-20 hydrogenated castor oil; PEG-25 hydrogenated castor oil; PEG-30 hydrogenated castor oil; PEG-35 hydrogenated castor oil; PEG-40 hydrogenated castor oil; PEG-45 hydrogenated castor oil; PEG-50 hydrogenated castor oil; PEG-54 hydrogenated castor oil; PEG-55 hydrogenated castor oil; PEG-60 hydrogenated castor oil; PEG-80 hydrogenated castor oil; PEG-100 hydrogenated castor oil; PEG-200 hydrogenated castor oil, and a mixture of two or more of these hydrogenated castor oils, the at least one ethoxylated hydrogenated castor oil more preferably being PEG-40 hydrogenated castor oil;
   wherein the at least one ethoxylated hydrogenated castor oil is admixed by a method comprising at least one sequence of homogenizing and mixing.

Yet further, the present invention relates to the pharmaceutical composition described above or the pharmaceutical composition obtainable or obtained by the process as described above for use in a method of treating or preventing fungal infections in mammals in need of such treating or preventing such infections.

Still further, the present invention relates to the use of a combination of crystalline posaconazole, preferably at least 90 weight-%, preferably at least 95 weight-%, more preferably at least 98 weight-% thereof being present as crystalline form IV, having an X-ray powder diffraction pattern comprising peaks at 2-theta angles of about 3.2°±0.2°, 6.6°±0.2°, 10.9°±0.2°, 16.9°±0.2°, 18.4°±0.2° and 25.1°±0.2°, measured with Cu—K alpha_{1,2} radiation, and/or having an attenuated total reflectance infrared spectrum comprising absorption bands at wavenumbers of about 3647 cm⁻¹±2 cm⁻¹, 3472 cm⁻¹±2 cm⁻¹, 2867 cm⁻¹±2 cm⁻¹, 1687 cm⁻¹±2 cm⁻¹, 1512 cm⁻¹±2 cm⁻¹, 1230 cm⁻¹±2 cm⁻¹, 1136 cm⁻¹±2 cm⁻¹, 916 cm⁻¹±2 cm⁻¹, 853 cm⁻¹±2 cm⁻¹, 819 cm⁻¹±2 cm⁻¹ and 681 cm⁻¹±2 cm⁻¹; and at least one ethoxylated hydrogenated castor oil for improving the long-term stability of liquid dosage forms comprising posaconazole with regard to the particle size distribution.

### DETAILED DESCRIPTION OF THE INVENTION

According to the present invention, the pharmaceutical composition comprises crystalline posaconazole and one or more non-ionic surfactants wherein at least one non-ionic surfactant is an ethoxylated hydrogenated castor oil.

### Crystalline Posaconazole

As to the crystalline posaconazole comprised in the pharmaceutical composition, all crystalline forms and mixtures of two or more of these forms are conceivable. Further, the crystalline posaconazole may also comprise amorphous posaconazole. The content of the crystalline posaconazole with regard to amorphous posaconazole is preferably less than 5 weight-%, preferably less than 1 weight-%, more preferably less than 0.1 weight-%, based on the total weight of the posaconazole comprised in the pharmaceutical composition. Most preferably, the crystalline posaconazole is essentially free, more preferably free of amorphous posaconazole. As to the conceivable crystalline forms of posaconazole, forms I, II, III, and IV can be mentioned by way of example. The preparation of crystalline form I of posaconazole is described, for example, in U.S. Pat. No. 6,958,337 B2, in example 2, column 13, lines 27 to 42, and in example 3, column 13, lines 44 to 58. The preparation of crystalline form II of posaconazole is described, for example, in U.S. Pat. No. 6,958,337 B2, in example 4, column 13, line 60 to column 14, line 7, and in example 5, column 14, lines 8 to 18. The preparation of crystalline form III of posaconazole is described, for example, in U.S. Pat. No. 6,958,337 B2, in example 6, column 14, lines 20 to 31. The preparation of crystalline form IV of posaconazole is described, for example, in WO 2010/000668 A1, in particular in example 1, page 21, line 5 to page 23, line 14; in example 2, page 23, lines 18 to 25 referring, reading the starting material, to example 6 described in U.S. Pat. No. 6,958,337 B2, column 14, lines 20 to 31; in example 3, page 23, lines 29 to 32; in example 4, page 24, lines 3 to 11. Therefore, according to conceivable embodiments of the present invention, the crystalline posaconazole comprised in the pharmaceutical composition may be pure crystalline form I, pure crystalline form II, pure crystalline form III, pure crystalline form IV, a mixture of forms I and II, a mixture of forms I and III, a mixture of forms I and IV, a mixture of forms II and III, a mixture of forms II and IV, a mixture of from III and IV, a mixture of forms I, II and III, a mixture of forms I, II and IV, a mixture of forms I, III and IV, a mixture of form II, III, and IV, and a mixture of forms I, II, III and IV. Preferably, the crystalline posaconazole comprised in the pharmaceutical composition comprises crystalline form IV of posaconazole.

According to preferred embodiments of the present invention, the crystalline posaconazole comprised in the pharmaceutical composition comprises at least 90 weight-%, more preferably at least 95 weight-%, more preferably at least 98 weight-%, more preferably at least 99 weight-%, more preferably at least 99.9 weight-%, more preferably at least 99.99 weight-%, based on the total weight of the posaconazole comprised in the pharmaceutical composition, of crystalline form IV of posaconazole. For example, the crystalline posaconazole comprised in the pharmaceutical composition may essentially consist of crystalline form IV, or consists of crystalline form IV.

With regard to the preparation of crystalline form IV of posaconazole, reference is made to the specific disclosure of WO 2010/000668 A1 discussed above. With regard to the characterization of crystalline form IV of posaconazole, reference is made to the specific disclosure of WO 2010/000668 A1; in particular, the general X-ray powder diffraction pattern of claim 1, page 28, lines 7 to 9, and the detailed X-ray powder diffraction pattern on page 22, Table 1; the X-ray powder diffraction pattern shown in FIG. 1; the attenuated total reflectance infrared spectrum according to claim 3, page 28, lines 15 to 19; the attenuated total reflectance infrared spectrum shown in FIG. 2; the differential scanning calorimetry curve shown in FIG. 3; the water content according to claim 6, page 28, lines 28 and 29.

Therefore, the present invention relates to the pharmaceutical composition described above, wherein at least 90 weight-%, preferably at least 95 weight-%, more preferably at least 98 weight-% of the posaconazole comprised in the pharmaceutical composition are present as crystalline form IV, having an X-ray powder diffraction pattern comprising peaks at 2-theta angles of about 3.2°±0.2°, 6.6°±0.2°, 10.9°±0.2°, 16.9°±0.2°, 18.4°±0.2° and 25.1°±0.2°, measured with Cu—K alpha_{1,2} radiation, and/or having an attenuated total reflectance infrared spectrum comprising absorption bands at wavenumbers of about 3647 cm⁻¹±2 cm⁻¹, 3472 cm⁻¹±2 cm⁻¹, 2867 cm⁻¹±2 cm⁻¹, 1687 cm⁻¹±2 cm⁻¹, 1512 cm⁻¹±2 cm⁻¹, 1230 cm⁻¹±2 cm⁻¹, 1136 cm⁻¹±2 cm⁻¹, 916 cm⁻¹±2 cm⁻¹, 853 cm⁻¹±2 cm⁻¹, 819 cm⁻¹±2 cm⁻¹ and 681 cm⁻¹±2 cm⁻¹.

### Ethoxylated Hydrogenated Castor Oil

According to the present invention, the pharmaceutical composition comprises at least one ethoxylated hydrogenated castor oil.

Castor oil is a vegetable oil obtained from the castor bean. It is a triglyceride wherein a certain percentage, usually about 90 percent, of fatty acid chains are ricinoleic acid. Ricinoleic acid is a monounsaturated, 18-carbon fatty acid which has a hydroxyl functional group on the 12th carbon atom. Oleic and linoleic acids are usually the other significant components of the triglycerides of the castor oil. A typical average composition of castor oil seed with respect to the fatty acid chains is given in the following table:

| Acid Name | Average Percentage Range/weight-% |
|---|---|
| Ricinoleic Acid | 95 to 85 |
| Oleic Acid | 6 to 2 |
| Linoleic Acid | 5 to 1 |
| Linolenic Acid | 1 to 0.5 |
| Stearic Acid | 1 to 0.5 |
| Palmitic Acid | 1 to 0.5 |
| Dihydroxystearic Acid | 0.5 to 0.3 |
| Others | 0.5 to 0.2 |

Hydrogenated castor oil refers to a castor oil of which a certain percentage, preferably essentially all of the carbon-carbon double bonds contained in the fatty acid residues in the triglycerides are hydrogenated. Ethoxlyated hydrogenated castor oil refers to a hydrogenated castor oil which is obtained by reacting the hydrogenated castor oil via the hydroxyl groups with ethylene oxide. Conventionally, if 1 mole of hydrogenated castor oil is reacted with x mole of ethylene oxide, the resulting product is referred to as “PEG-x hydrogenated castor oil” wherein “PEG” stands for polyethylene glycol. For example, PEG-x hydrogenated castor oil with x=40, i.e. PEG-40 hydrogenated castor oil, is obtained by reacting 1 mole of hydrogenated castor oil with 40 moles of ethylene oxide. Ethoxlyated hydrogenated castor oils are also commercially available. PEG-40 hydrogenated castor oil, for example, is available as Cremophor® RH 40 from BASF (CAS-Nr. 61788-85-0) or Kolliphor® RH 40 from Sigma-Aldrich, also referred to as “Polyoxyl 40 hydrogenated castor oil” or “Macrogolglycerol Hydroxystearate”.

Generally, it is conceivable that the pharmaceutical composition of the present invention contains one or more different ethoxylated hydrogenated castor oils. Preferably, PEG-x hydrogenated castor oils are employed wherein x is in the range of from 5 to 200. More preferably, the at least one ethoxylated hydrogenated castor oil is selected from the group consisting of PEG-5 hydrogenated castor oil; PEG-7 hydrogenated castor oil; PEG-16 hydrogenated castor oil; PEG-20 hydrogenated castor oil; PEG-25 hydrogenated castor oil; PEG-30 hydrogenated castor oil; PEG-35 hydrogenated castor oil; PEG-40 hydrogenated castor oil; PEG-45 hydrogenated castor oil; PEG-50 hydrogenated castor oil; PEG-54 hydrogenated castor oil; PEG-55 hydrogenated castor oil; PEG-60 hydrogenated castor oil; PEG-80 hydrogenated castor oil; PEG-100 hydrogenated castor oil; PEG-200 hydrogenated castor oil, and a mixture of two or more of these hydrogenated castor oils. Most preferably, at least one of the ethoxylated hydrogenated castor oils is PEG-40 hydrogenated castor oil; even more preferably, the pharmaceutical composition of the present invention contains exactly one ethoxylated hydrogenated castor oil, most preferably PEG-40 hydrogenated castor oil.

Further, it may be conceivable to employ, instead of or in addition to the at least one ethoxylated hydrogenated castor oil, at least one ethoxylated glyceride which is selected from the group consisting of PEG-6 caprylic/capric glycerides PEG-8 caprylic/capric clycerides; PEG-2 castor oil; PEG-3 castor oil; PEG-4 Castor Oil; PEG-5 Castor Oil; PEG-8 Castor Oil; PEG-9 Castor Oil; PEG-10 Castor Oil; PEG-11 Castor Oil; PEG-15 Castor Oil; PEG-20 Castor Oil; PEG-25 Castor Oil; PEG-30 Castor Oil; PEG-33 Castor Oil; PEG-35 Castor Oil; PEG-36 Castor Oil; PEG-40 Castor Oil; PEG-50 Castor Oil; PEG-54 Castor Oil; PEG-55 Castor Oil; PEG-60 Castor Oil; PEG-100 Castor Oil; PEG-200 Castor Oil; PEG-18 Castor Oil Dioleate; PEG-60 Corn Glycerides; PEG-20 Evening Primrose Glycerides; PEG-60 Evening Primrose Glycerides; PEG-7 Glyceryl Cocoate; PEG-30 Glyceryl Cocoate; PEG-78 Glyceryl Cocoate; PEG-80 Glyceryl Cocoate; PEG-12 Glyceryl Dioleate; PEG-15 Glyceryl Isostearate; PEG-20 Glyceryl Isostearate; PEG-30 Glyceryl Isostearate; PEG-60 Glyceryl Isostearate; PEG-12 Glyceryl Laurate; PEG-20 Glyceryl Laurate; PEG-23 Glyceryl Laurate; PEG-30 Glyceryl Laurate; PEG-10 Glyceryl Oleate; PEG-15 Glyceryl Oleate; PEG-30 Glyceryl Oleate; PEG-20 Glyceryl Ricinoleate; PEG-5 Glyceryl Sesquioleate; PEG-5 Glyceryl Stearate; PEG-10 Glyceryl Stearate; PEG-25 Glyceryl Stearate; PEG-30 Glyceryl Stearate; PEG-120 Glyceryl Stearate; PEG-200 Glyceryl Stearate; PEG-28 Glyceryl Tallowate; PEG-80 Glyceryl Tallowate; PEG-200 Glyceryl Tallowate; PEG-5 Glyceryl Triisostearate; PEG-40 Hydrogenated Castor Oil PCA Isosterate; PEG-5 Hydrogenated Corn Glycerides; PEG-8 Hydrogenated Fish Glycerides; and a mixture of two or more of these ethoxylated glycerides. For example, a conceivable castor oil is a commercial product sold as Cremophor® EL Castor Oil from BASF, CAS number 61791-12-6.

In particular for liquid pharmaceutical compositions, it was surprisingly found that the presence of the at least one ethoxylated hydrogenated castor oil has an advantageous influence on the long-term stability of the liquid compositions with respect to the particle size distribution. In particular for liquid compositions comprising the crystalline posaconazole in the form of small particles, it was found that the particle size distribution, characterized by the d(0.1), d(0.5) and d(0.9) values, does not change significantly if in the liquid composition, at least one ethoxylated hydrogenated castor oil, preferably PEG-40 hydrogenated castor oil is comprised. Small particles referred to hereinabove are characterized, for example, by a d(0.1) value of at most 5 micrometer, preferably at most 4 micrometer, more preferably at most 3 micrometer, more preferably in the range of from 1 to 3 micrometer, more preferably of from 1 to 2 micrometer; by a d(0.5) value of at most 10 micrometer, preferably at most 7 micrometer, more preferably at most 5 micrometer, more preferably in the range of from 3 to 5 micrometer, more preferably of from 3 to 4 micrometer; and by a d(0.9) value of at most 20 micrometer, preferably at most 15 micrometer, more preferably at most 11 micrometer, more preferably in the range of from 8 to 11 micrometer, more preferably of from 8 to 9 micrometer.

Therefore, the present invention also relates to the pharmaceutical composition described above, having a particle size distribution characterized by a d(0.1) value in the range of from 1 to 3, preferably from 1 to 2 micrometer, a d(0.5) value in the range of from 3 to 5, preferably from 3 to 4 micrometer, and a d(0.9) value in the range of from 8 to 11, preferably from 8 to 9 micrometer.

Preferably, such pharmaceutical composition comprising the posaconazole particles having a small size, preferably according to a particle size distribution characterized by a d(0.1) value in the range of from 1 to 3, preferably from 1 to 2 micrometer, a d(0.5) value in the range of from 3 to 5, preferably from 3 to 4 micrometer, and a d(0.9) value in the range of from 8 to 11, preferably from 8 to 9 micrometer, exhibits a long-term stability with regard to the particle size distribution of at least 6 months, preferably of at least 12 months, more preferably of at least 18 months, more preferably of at least 24 months, more preferably of at least 36 months, wherein the long-term stability with regard to the particle size distribution is characterized in a change in the d(0.1) value of at most 10%, preferably of at most 7%, in a change in the d(0.5) value of at most 10%, preferably of at most 5%, and in a change in the d(0.9) value of at most 15%, preferably of at most 12%.

Generally, there are no specific restrictions concerning the amount of the at least one ethoxylated hydrogenated castor oil comprised in the pharmaceutical composition. However, it was found that the ratio of the weight of the at least one ethoxylated hydrogenated castor oil relative to the weight of posaconazole is preferably in the range of from 1.5:1 to 8.5:1, preferably from 2.3:1 to 7.2:1, more preferably from 3.6:1 to 5.1:1, more preferably from 4.2:1 to 4.5:1.

As mentioned above, the pharmaceutical composition is preferably a liquid composition. Therefore, it is preferred that the pharmaceutical composition is a liquid dosage form. Even more preferably, it is an oral liquid dosage form. Such liquid compositions comprise, for example, liquid suspensions and liquid dispersions, with liquid suspensions being preferred. Even more preferred compositions additionally comprise water, i.e. are aqueous compositions, in particular aqueous suspensions. While generally, the amount of water comprised in the suspension is not subject to any specific restrictions, preferred pharmaceutical compositions of the present invention, preferably liquid suspensions, are characterized by a ratio of the weight of the water relative to the weight of posaconazole in the range of from 10:1 to 20:1, preferably from 12:1 to 15:1, more preferably from 13:1 to 14:1.

Preferably, the pharmaceutical compositions of the present invention additionally comprise at least one further non-ionic surfactant. Such suitable non-ionic surfactants include, but are not limited to, block copolymers of ethylene oxide and propylene oxide, polyoxyethylene ethers of saturated or unsaturated C₈ to C₂₀ acids, and polyvinyl alcohols or sorbitan esters of saturated or unsaturated C₁₀ to C₂₀ acids. Preferably, the non-ionic surfactant additionally comprised in the pharmaceutical composition of the present invention is a sorbitan ester of a saturated or unsaturated C₁₀ to C₂₀ acid, and more preferably, the additionally comprised non-ionic surfactant is a fatty acid ester of sorbitan selected from sorbitan monolaurate, sorbitan monooleate, sorbitan sesquioleate, sorbitan trioleate, sorbitan monopalmitate, sorbitan monostearate and sorbitan tristearate, or mixtures thereof. Suitable sorbitan esters include, but are not limited to, Polysorbate 20, Polysorbate 40, Polysorbate 60, Polysorbate 65, Polysorbate 80, Polysorbate 85, Sorbitan Monolaurate, Sorbitan Monooleate, Sorbitan Monopalmitate, Sorbitan Monostearate, Sorbitan Sesquioleate, Sorbitan Trioleate, Sorbitan Tristearate, and mixtures of two or more thereof. Most preferably, the pharmaceutical composition of the present invention additionally comprises as additional non-ionic surfactant at least Polysorbate 80. More preferably, the pharmaceutical composition of the present invention additionally comprises exactly one non-ionic surfactant, which is preferably Polysorbate 80. Polysorbate 80 is commercially available i.a. under the tradename Tween® 80 from ICI.

Therefore, the present invention also relates to the pharmaceutical composition as described above, additionally comprising at least one further non-ionic surfactant selected from the group consisting of polyoxyethylene derivatives of sorbitan esters of saturated C₁₀ to C₂₀ acids, polyoxyethylene derivatives of sorbitan esters of unsaturated C₁₀ to C₂₀ acids, and mixtures of two or more thereof.

Generally, there are no specific restrictions concerning the amount of the at least one additional non-ionic surfactant comprised in the pharmaceutical composition. However, it was found that the ratio of the weight of the at least one further non-ionic surfactant relative to the weight of posaconazole is preferably in the range of from 0.05:1 to 1:1, more preferably from 0.1:1 to 0.5:1, more preferably from 0.2:1 to 0.3:1, more preferably from 0.22:1 to 0.28:1.

According to an especially preferred embodiment of the present invention, the pharmaceutical composition comprises exactly two non-ionic surfactants, one ethoxylated hydrogenated castor oil, preferably PEG-40 hydrogenated castor oil, and one fatty acid ester of sorbitan, preferably Polysorbate 80.

Preferably, the pharmaceutical composition of the present invention additionally comprises at least one buffering agent. The buffering agents suitable for the pharmaceutical composition of the present invention are those which allow to maintain the pH of the pharmaceutical composition, preferably the liquid suspension, in the range of from about 4 to about 6, preferably of from about 4.3 to 5.0, and most preferably of about 4.5 to about 4.7. The use of the buffering agent sodium citrate and citric acid is preferred. Generally, there are no specific restrictions concerning the amount of the at least one buffering agent comprised in the pharmaceutical composition. However, it was found that the ratio of the weight of the at least one buffering agent relative to the weight of posaconazole is preferably in the range of from 0.05:1 to 0.2:1, more preferably from 0.07:1 to 0.15:1, more preferably from 0.08:1 to 0.1:1.

Preferably, the pharmaceutical composition of the present invention additionally comprises at least one flavoring agent. Preferred are those flavoring agents approved by FDA for use in sweetened pharmaceuticals, foods, candies, beverages and the like. Preferably, these flavoring agents impart flavors such as grape, cherry, citrus, peach, strawberry, bubble gum, peppermint, or others. Most preferably, the pharmaceutical composition of the present invention comprises an agent imparting cherry flavor. Generally, there are no specific restrictions concerning the amount of the at least one flavoring agent comprised in the pharmaceutical composition. However, it was found that the ratio of the weight of the at least one flavoring agent relative to the weight of posaconazole is preferably in the range of from 0.15:1 to 0.5:1, more preferably from 0.16:1 to 0.3:1, more preferably from 0.17:1 to 0.2:1.

Preferably, the pharmaceutical composition of the present invention additionally comprises at least one thickening agent. Preferred thickening agents according to the present invention include any commercially available agent useful for such purpose such as xanthan gum, liquid sugars such as liquid glucose, glucose in the form of corn syrup solids, starches, celluloses and mixtures of two or more thereof. More preferred is a combination of xanthan gum and glucose. Generally, there are no specific restrictions concerning the amount of the at least one thickening agent comprised in the pharmaceutical composition. However, it was found that the ratio of the weight of the at least one thickening agent relative to the weight of posaconazole is preferably in the range of from 5:1 to 8.5:1, more preferably from 6:1 to 7.5:1, more preferably from 6.5:1 to 7:1.

Therefore, the present invention relates to the pharmaceutical composition as described above, additionally comprising at least one buffering agent and/or at least one flavoring agent and/or at least one thickening agent, preferably at least one buffering agent and at least one flavoring agent and at least one thickening agent,

wherein the ratio of the weight of the at least one buffering agent relative to the weight of posaconazole is preferably in the range of from 0.05:1 to 0.2:1, more preferably from 0.07:1 to 0.15:1, more preferably from 0.08:1 to 0.1:1, the at least one buffering agent preferably being a mixture of sodium citrate dihydrate and citric acid monohydrate;
wherein the ratio of the weight of the at least one flavoring agent relative to the weight of posaconazole is preferably in the range of from 0.15:1 to 0.5:1, more preferably from 0.16:1 to 0.3:1, more preferably from 0.17:1 to 0.2:1, the at least one flavoring agent preferably being cherry flavor; and wherein the ratio of the weight of the at least one thickening agent relative to the weight of posaconazole is preferably in the range of from 5:1 to 8.5:1, more preferably from 6:1 to 7.5:1, more preferably from 6.5:1 to 7:1, the at least one thickening agent preferably being a polysaccharide, more preferably being selected from the group consisting of glucose, xanthan gum, and a mixture thereof.

Further, the pharmaceutical composition of the present invention may comprise other suitable additives such as at least one antifoaming agent, at least one preservative, at least one additional solvent, at least one carrier, at least one cap anti-locking agent, at least one opacifier agent, and a mixture of two or more thereof.

Preferred carriers include, but are not limited to, glycerin (glycerol). Generally, there are no specific restrictions concerning the amount of the at least one carrier comprised in the pharmaceutical composition. However, it was found that the ratio of the weight of the at least one carrier, relative to the weight of posaconazole is preferably in the range of from 0.5:1 to 10:1, more preferably from 1:1 to 5:1, more preferably from 2:1 to 3:1, more preferably from 2.3:1 to 2.7:1.

Preferred anti-foaming agents include, but are not limited to, commercially available agents useful for such purpose including the methylated linear siloxane polymers end blocked with trimethylsiloxyl units such as dimethicone and simethicone, as well as mixtures of dimethicone with an average chain length of 200 to 250 dimethylsiloxane units, and silica gel, with simethicone being most preferred. Generally, there are no specific restrictions concerning the amount of the at least one anti-foaming agent comprised in the pharmaceutical composition. However, it was found that the ratio of the weight of the at least one anti-foaming agent relative to the weight of posaconazole is preferably in the range of from 0.01:1 to 0.5:1, more preferably from 0.04:1 to 0.2:1, more preferably from 0.06:1 to 0.09:1.

Preferred preservatives include, but are not limited to, water soluble preservatives such as sodium benzoate, sodium citrate and benzalkonium chloride as well as other pharmaceutical acceptable water soluble preservatives, with sodium benzoate being most preferred. Generally, there are no specific restrictions concerning the amount of the at least one preservative comprised in the pharmaceutical composition. However, it was found that the ratio of the weight of the at least one preservative to the weight of posaconazole is preferably in the range of from 0.01:1 to 0.2:1, more preferably from 0.02:1 to 0.1:1, more preferably from 0.03:1 to 0.07:1.

Preferred opacifier agents include, but are not limited to, pharmaceutically acceptable metal oxides, with titanium dioxide being most preferred. Generally, there are no specific restrictions concerning the amount of the at least one opacifier agent comprised in the pharmaceutical composition. However, it was found that the ratio of the weight of the at least one opacifier agent relative to the weight of posaconazole is preferably in the range of from 0.02:1 to 0.4:1, more preferably from 0.04:1 to 0.2:1, more preferably from 0.06:1 to 0.15:1.

Therefore, according to an especially preferred embodiment, the present invention relates to a pharmaceutical composition, preferably a liquid suspension, comprising crystalline posaconazole, at least 90 weight-%, preferably at least 95 weight-%, more preferably at least 98 weight-%, based on the total weight of the posaconazole comprised in the pharmaceutical composition, being posaconazole of crystalline form IV;

at least one ethoxylated hydrogenated castor oil wherein the ratio of the weight of the at least one ethoxylated hydrogenated castor oil relative to the weight of posaconazole is preferably in the range of from 1.5:1 to 8.5:1, preferably from 2.3:1 to 7.2:1, more preferably from 3.6:1 to 5.1:1, more preferably from 4.2:1 to 4.5:1, the ethoxylated hydrogenated castor oil preferably being PEG-40 hydrogenated castor oil;
at least one polyoxyethylene derivative of sorbitan esters of saturated C₁₀ to C₂₀ acids, polyoxyethylene derivatives of sorbitan esters of unsaturated C₁₀ to C₂₀ acids, and mixtures of two or more thereof, wherein the ratio of the weight of the at least one derivative relative to the weight of posaconazole is preferably in the range of from 0.05:1 to 1:1, more preferably from 0.1:1 to 0.5:1, more preferably from 0.2:1 to 0.3:1, more preferably from 0.22:1 to 0.28:1, the derivative preferably being Polysorbate 80;
at least one buffering agent, wherein the at least one buffering agent relative to the weight of posaconazole is preferably in the range of from 0.05:1 to 0.2:1, more preferably from 0.07:1 to 0.15:1, more preferably from 0.08:1 to 0.1:1, the at least one buffering agent preferably being a mixture of sodium citrate dihydrate and citric acid monohydrate;
at least one flavoring agent, wherein the ratio of the weight of the at least one flavoring agent relative to the weight of posaconazole is preferably in the range of from 0.15:1 to 0.5:1, more preferably from 0.16:1 to 0.3:1, more preferably from 0.17:1 to 0.2:1, the flavoring agent preferably being cherry flavor;
at least one thickening agent, wherein the ratio of the weight of the at least one thickening agent relative to the weight of posaconazole is preferably in the range of from 5:1 to 8.5:1, more preferably from 6:1 to 7.5:1, more preferably from 6.5:1 to 7:1, the at least one thickening agent preferably being a polysaccharide, more preferably being selected from the group consisting of glucose, xanthan gum, and a mixture thereof;
at least one carrier, wherein the ratio of the weight of the at least one carrier relative to the weight of posaconazole is preferably in the range of from 0.5:1 to 10:1, more preferably from 1:1 to 5:1, more preferably from 2:1 to 3:1, more preferably from 2.3:1 to 2.7:1, the carrier preferably being glycerin;
at least one anti-foaming agent, wherein the ratio of the weight of the at least one anti-foaming agent relative to the weight of posaconazole is preferably in the range of from 0.01:1 to 0.5:1, more preferably from 0.04:1 to 0.2:1, more preferably from 0.06:1 to 0.09:1, the anti-foaming agent preferably being simethicone;
at least one preservative, wherein the ratio of the weight of the at least one preservative to the weight of posaconazole is preferably in the range of from 0.01:1 to 0.2:1, more preferably from 0.02:1 to 0.1:1, more preferably from 0.03:1 to 0.07:1, the preservative preferably being sodium benzoate;
at least one opacifier agent, wherein the ratio of the weight of the at least one opacifier agent relative to the weight of posaconazole is preferably in the range of from 0.02:1 to 0.4:1, more preferably from 0.04:1 to 0.2:1, more preferably from 0.06:1 to 0.15:1, the opacifier agent preferably being titanium dioxide; and
water, wherein the ratio of the weight of the water relative to the weight of posaconazole is preferably in the range of from 10:1 to 20:1, more preferably from 12:1 to 15:1, more preferably from 13:1 to 14:1.

An especially preferred pharmaceutical composition according to the present invention has the following composition:

from 3 to 4 weight-% of crystalline posaconazole, at least 90 weight-%, preferably at least 95 weight-%, more preferably at least 98 weight-%, based on the total weight of the posaconazole comprised in the pharmaceutical composition, being posaconazole of crystalline form IV;
from 14.5 to 15.5 weight-% of PEG-40 hydrogenated castor oil;
from 0.75 to 1 weight-% of Polysorbate 80;
from 0.28 to 0.35 weight-% of a mixture of sodium citrate dihydrate and citric acid monohydrate, preferably having a weight ratio of sodium citrate dihydrate relative to citric acid monohydrate of from 1:4 to 1:6, preferably about 1:5;
from 0.6 to 0.7 weight-% of cherry flavor;
from 22 to 25 weight-% of a mixture of glucose and xanthan gum, preferably having a weight ratio of glucose relative to xanthan gum of 250:1 to 300:1, preferably from 270:1 to 280:1, more preferably about 276:1;
from 7.9 to 9.3 weight-% of glycerin;
from 0.2 to 0.3 weight-% of simethicone;
from 0.1 to 0.2 weight-% of sodium benzoate;
from 0.2 to 0.5 weight-% of titanium dioxide;
from 45 to 48 weight-% of water;
wherein the weight-% values of the individual compounds add up to 100%.

### Process

As far as the process for the preparation of the pharmaceutical composition of the present invention is concerned, no specific restrictions exist. Generally, the compounds are admixed in a suitable sequence of steps, wherein mixtures, if necessary, can be suitably homogenized. The temperatures at which mixing and/or homogenization is carried out can be suitably chosen and usually are in the range of from 20 to 60° C.

Generally, the process comprises
(aa) providing crystalline posaconazole, wherein preferably at least 90 weight-%, more preferably at least 95 weight-%, more preferably at least 98 weight-% of the posaconazole are present as crystalline form IV having an X-ray powder diffraction pattern comprising peaks at 2-theta angles of about 3.2°±0.2°, 6.6°±0.2°, 10.9°±0.2°, 16.9°±0.2°, 18.4°±0.2° and 25.1°±0.2°, measured with Cu—K alpha_{1,2} radiation, and/or having an attenuated total reflectance infrared spectrum comprising absorption bands at wavenumbers of about 3647 cm⁻¹±2 cm⁻¹, 3472 cm⁻¹±2 cm⁻¹, 2867 cm⁻¹±2 cm⁻¹, 1687 cm⁻¹±2 cm⁻¹, 1512 cm⁻¹±2 cm⁻¹, 1230 cm⁻¹±2 cm⁻¹, 1136 cm⁻¹±2 cm⁻¹, 916 cm⁻¹±2 cm⁻¹, 853 cm⁻¹±2 cm⁻¹, 819 cm⁻¹±2 cm⁻¹ and 681 cm⁻¹±2 cm⁻¹;
(bb) mixing one or more non-ionic surfactants with the posaconazole provided in (aa), wherein at least one non-ionic surfactant is an ethoxylated hydrogenated castor oil, the at least one ethoxylated hydrogenated castor oil preferably being selected from the group consisting of PEG-5 hydrogenated castor oil; PEG-7 hydrogenated castor oil; PEG-16 hydrogenated castor oil; PEG-20 hydrogenated castor oil; PEG-25 hydrogenated castor oil; PEG-30 hydrogenated castor oil; PEG-35 hydrogenated castor oil; PEG-40 hydrogenated castor oil; PEG-45 hydrogenated castor oil; PEG-50 hydrogenated castor oil; PEG-54 hydrogenated castor oil; PEG-55 hydrogenated castor oil; PEG-60 hydrogenated castor oil; PEG-80 hydrogenated castor oil; PEG-100 hydrogenated castor oil; PEG-200 hydrogenated castor oil, and a mixture of two or more of these hydrogenated castor oils, the at least one ethoxylated hydrogenated castor oil more preferably being PEG-40 hydrogenated castor oil.

Preferably, the at least one ethoxylated hydrogenated castor oil is admixed by a method comprising at least one sequence of homogenizing and mixing. In the course of such a sequence, the at least one ethoxylated hydrogenated castor oil is added, and the resulting mixture is homogenized, preferably by using an homogenizing apparatus such as a process vessel Fryma VME 120/95, followed by normal mixing. Preferably, homogenizing and/or mixing, preferably homogenizing and mixing are carried out a temperature in the range of from 20 to 75° C., preferably from 35 to 70° C., more preferably from 50 to 70° C., more preferably from 55 to 65° C. such as about 60° C.

Surprisingly, it was found that employing the ethoxylated hydrogenated castor oil in the process, liquid suspensions were obtained which turned out to exhibit an excellent long-term stability with respect to the particle size distribution, in particular for liquid suspension comprising crystalline posaconazole having a small particle size as defined above. Moreover, it was found that the particle size distribution is essentially constant in case the ethoxylated hydrogenated castor oil is used, even if the particles are comparatively small.

Therefore, the present invention also relates to the use of a combination of crystalline posaconazole, preferably at least 90 weight-%, preferably at least 95 weight-%, more preferably at least 98 weight-% thereof being present as crystalline form IV, having an X-ray powder diffraction pattern comprising peaks at 2-theta angles of about 3.2°±0.2°, 6.6°±0.2°, 10.9°±0.2°, 16.9°±0.2°, 18.4°±0.2° and 25.1°±0.2°, measured with Cu—K alpha_{1,2} radiation, and/or having an attenuated total reflectance infrared spectrum comprising absorption bands at wavenumbers of about 3647 cm⁻¹±2 cm⁻¹, 3472 cm⁻¹±2 cm⁻¹, 2867 cm⁻¹±2 cm⁻¹, 1687 cm⁻¹±2 cm⁻¹, 1512 cm⁻¹±2 cm⁻¹, 1230 cm⁻¹±2 cm⁻¹, 1136 cm⁻¹±2 cm⁻¹, 916 cm⁻¹±2 cm⁻¹, 853 cm⁻¹±2 cm⁻¹, 819 cm⁻¹±2 cm⁻¹ and 681 cm⁻¹±2 cm⁻¹; and at least one ethoxylated hydrogenated castor oil, preferably PEG-40 hydrogenated castor oil, for improving the long-term stability of liquid dosage forms comprising posaconazole with regard to the particle size distribution. As far as preferred particle size distributions are concerned, reference is made to the particle size distributions as defined above.

As already described in WO2010/000668 A1, posaconazole having crystalline form IV can be prepared having a small particle size, wherein no micro fluidization, preferably no micronization has to be carried out in order to obtain a crystalline material characterized by small particle sizes.

Furthermore, the addition of the ethoxylated hydrogenated castor oil was found to facilitate the manufacturing process to achieve a homogenous suspension with finley dispersed posaconazole particles, which advantage is confirmed by a particle size distribution characterized by small particles as described in Example 3 hereinunder. Surprisingly, this advantageous particle size distribution with particles which are even smaller than those of known posaconazole suspensions, e.g. Noxafil®, can be obtained by the inventive process by applying a much lower input of energy (homogenization) than the prior art process (microfluidization) for preparing e.g. Noxafil®.

Therefore, the present invention also relates to the process as described above, wherein during the entire process for the preparation of the pharmaceutical composition including the providing in (aa), no microfluidization, preferably no micronization is carried out.

Further, the present invention relates to the pharmaceutical composition as described above or the pharmaceutical composition obtainable or obtained by the process as described above for use in a method of treating or preventing fungal infections in mammals in need of such treating or preventing such infections.

The present invention is illustrated by the following examples.

### Examples

### 1. Preparation of Crystalline Form IV of Posaconazole

Posaconazole was prepared according to the method disclosed in WO 2011/144653 A1, Example 5, on page 74, line 20, to page 76, line 14.

A mixture of 8.0 kg of the thereby obtained posaconazole and of 126.4 kg of methanol was heated to 65±2° C. whereby a clear solution was obtained. The solution was filtrated and cooled to 35±2° C. followed by cooling from 35±2° C. to 15±2° C. within about 2 hours whereupon crystallization occurred. The obtained suspension was further stirred at 15±2° C. for about 1 hour before it was further cooled to −5±2° C. and kept at the same temperature for additional 2 hours. The solid material was isolated by centrifugation, and the wet cake was added back into the empty agitation vessel. 0.56 kg of posaconazole form IV seeds (obtained according to the method described in example 2 of WO 2010/000668 A1, page 23, lines 16 to 25), 160.0 kg of water and 30.4 kg of methanol were added, and the obtained suspension was heated to 43±2° C. and stirred at this temperature for about 6 days whereby posaconazole crystalline form IV was obtained in polymorphically pure form (as confirmed by XRPD according to the method disclosed in WO 2010/000668, on page 19, lines 17 to 24). Thereafter the suspension was cooled to 25±2° C. and kept at the same temperature for about 2 hours before the solid material was isolated by centrifugation and dried at 40±2° C. under vacuum for about 16.5 hours to obtain 7.1 kg of polymorphically pure form IV of posaconazole.

### 2. Preparation of a Pharmaceutical Composition in the Form of a Liquid Suspension

A liquid suspension was prepared having the following composition:

| Ingredient | Quantity/(g/5 mL) | % (w/w) |
|---|---|---|
| Posaconazole Crystalline Form IV | 0.20000 | 3.45 |
| obtained according to Example 1 above | | |
| Polysorbate 80 | 0.05000 | 0.86 |
| Simethicone | 0.01500 | 0.26 |
| Sodium Benzoate | 0.01000 | 0.17 |
| Sodium Citrate Dihydrate | 0.00300 | 0.05 |
| Citric Acid Monohydrate | 0.01500 | 0.26 |
| Glycerol | 0.50000 | 8.62 |
| Xanthan Gum | 0.00500 | 0.09 |
| Corn Syrup Solids | 1.38250 | 24.83 |
| Titanium Dioxide | 0.02000 | 0.34 |
| Polyoxyl 40 Hydrogenated Castor Oil | 0.87000 | 15.00 |
| Cherry Flavor | 0.04000 | 0.69 |
| Purified Water | 2.68950 | 45.38 |
| Total Mass: | 5.80000 g | 100.0 |

The polysorbate 80 (NF quality) and the simethicone (NF quality) were admixed with a portion of the purified water (USP quality) which had been heated to 50° C. The components were dissolved by mixing and emulsified by homogenization (Process Vessel Fryma VME 120/95, available from FrymaKoruma). After cooling to room temperature, the posaconazole prepared as described above was dispersed in this mixture at room temperature, and the resulting mixture was emulsified by homogenization (Process Vessel Fryma VME 120/95, available from FrymaKoruma). Thereafter, the sodium benzoate, the sodium dihydrate and the citric acid monohydrate (all NF quality) were admixed at room temperature. To this mixture, the glycerol (NF quality) was admixed. Then, a mixture of the corn syrup solids and the xanthan gum (both NF quality) were admixed, followed by admixing the titanium dioxide (NF quality) at room temperature.

In the next step, the mixture was heated to 60° C., and at 60° C., the PEG-40 hydrogenated castor oil (Cremophor® RH 40 purchased from BASF) was added, followed by homogenization for 120 min (Process Vessel Fryma VME 120/95, available from FrymaKoruma) and mixing for 120 min.

After cooling to room temperature, the cherry flavor was added, and the final portion of the purified water was added by mixing to obtain the above-described liquid suspension.

The obtained liquid suspension was filled in a 125 mL amber glass bottle having a screw pilfer proof N 28 and a child-resistant screw cap with a tamper-evident ring. The bottle was stored at room temperature.

### 3. Determination of the Particle Size Distribution and Comparison with Commercially Available Liquid Suspension Containing Posaconazole

3.1 The commercially available liquid suspension which was used for comparison reasons was Noxafil® (US) #0PSN505, stored at 25° C. at a relative humidity of 60%. In the following table, the composition of this commercially available suspension and the composition of the liquid suspension prepared according to the present invention are shown:

| | Noxafil ® (US) | According to |
|---|---|---|
| | #0PSN505 | present invention |
| Ingredient | mg/unit dose (5 mL) | mg/unit dose (5 mL) |
| Crystalline Posaconazole | Form I: 200.00 | Form IV: 200.00 |
| Polysorbate 80 | 50.00 | 50.00 |
| Simethicone | 15.00 | 15.00 |
| Sodium Benzoate | 10.00 | 10.00 |
| Sodium Citrate Dihydrate | 8.50 | 3.00 |
| Citric Acid Monohydrate | | 15.00 |
| Glycerol | 500.00 | 500.00 |
| Xanthan Gum | 15.00 | 5.00 |
| Liquid Glucose/Corn Syrup | 1750.00 | 1382.50 |
| Solids | | |
| Titanium Dioxide | 20.00 | 20.00 |
| Polyoxyl 40 Hydrogenated | — | 870.00 |
| Castor Oil | | |
| Cherry Flavor | 25.00 | 40.00 |

The particle size distribution (Malvern) was determined according to the USP 32 (2009) method <429> and EP 6 (2008) method 2.9.31 based on the diffraction of laser by particles using a Mastersizer 2000S liquid dispersion system without ultrasonication before the measurement. In particular, the following parameters were chosen:
Equipment: as described in the USP <429> method Ic/Ph. Eur. 2.5.32
   Laser diffraction particle sizer according to USP, Mastersizer 2000S liquid dispersion system
Instrument Settings: Measurement range 0.02-2000 micrometer
   Stirring speed 2000 to 5000 r.p.m.
   Obscuration 5-15%
   Data acquisition 12 s for background and sample
   Optical Model Mie evaluation, refractive index=1.54, absorbance=0.01
   Mathematical model general purpose, irregular shape
Data Evaluation: Measurement data were evaluated as volume size distribution; from this distribution, values for 10% [d(0.1)], 50% [d(0.5)], and 90% [d(0.9)] were determined.
3.2 The particle size distribution was determined as described above after 0, 3, 6, 9, 12, 18 and 24 months for inverted samples (bottle stored upside down) as follows:

| | d(0.1)/ | | d(0.5)/ | | d(0.9)/ | |
|---|---|---|---|---|---|---|
| | micrometer | | micrometer | | micrometer | |
| | | Inven- | | Inven- | | Inven- |
| Months | Noxafil ® | tion | Noxafil ® | tion | Noxafil ® | tion |
| 0 | 1.8 | 1.6 | 6.6 | 3.7 | 13.6 | 8.2 |
| 3 | 2.9 | 1.7 | 9.3 | 3.8 | 17.9 | 8.6 |
| 6 | n.d.*) | 1.7 | n.d.*) | 3.7 | n.d.*) | 8.4 |
| 9 | n.d.*) | 1.7 | n.d.*) | 3.8 | n.d.*) | 8.6 |
| 12 | 1.9 | 1.6 | 7.0 | 3.7 | 14.1 | 8.6 |
| 18 | n.d.*) | 1.6 | n.d.*) | 3.7 | n.d.*) | 8.6 |
| 24 | 1.9 | 1.7 | 6.0 | 3.7 | 12.2 | 8.0 |

*) not determined

First, it can be seen from the particle size distribution that the liquid suspension prepared according to the present invention contains considerably smaller particles, although throughout the whole preparation process, no micro fluidization, in particular no micronization was performed.

Second, it can be seen that the liquid suspensions according to the present invention are characterized by an excellent long-term stability with respect to the particle size distribution since even after 12 months, no change in the d(0.1) value is observable, and also after 3, 6, and 9 months, said change is less than 7%. Even after 24 months, the change in the d(0.1) value is only about 6.2%. As to the d(0.5) value, no change after 6, 12, 18 and 24 months can be observed, wherein after 3 and 9 months, the change is even only less than 3%. Also for the d(0.9) value, a very constant value is observed after 3, 9, 12 and 18 months and the change in the d(0.9) value after 6 months and after 24 months is only less than 3%. In contrast thereto, the d(0.1), d(0.5) and d(0.9) values of the commercially available product were quite inconstant over the observation period of 24 months. In particular after 3 months, a considerable increase in particle size was observed, with a change in d(0.1) of more than 60%, in d(0.5) of more than 40%, and in d(0.9) of more than 30%.

Therefore, it is shown that the pharmaceutical compositions of the present invention exhibit an excellent long-term stability with respect to the particle size distribution, in particular in combination with small particle sizes.
3.3 In a further test, the particle size distribution was determined after 0, 3, 6, 9, 12, and 18 months for non-inverted samples (normally stored bottle, not upside down) as follows:

| | d(0.1)/ | | d(0.5)/ | | d(0.9)/ | |
|---|---|---|---|---|---|---|
| | micrometer | | micrometer | | micrometer | |
| | | Inven- | | Inven- | | Inven- |
| Months | Noxafil ® | tion | Noxafil ® | tion | Noxafil ® | tion |
| 0 | 2.4 | 1.6 | 6.1 | 3.7 | 11.9 | 8.6 |
| 3 | 1.8 | 1.6 | 6.6 | 3.7 | 13.6 | 8.2 |
| 6 | n.d.*) | 1.7 | n.d.*) | 3.7 | n.d.*) | 8.2 |
| 9 | n.d.*) | 1.7 | n.d.*) | 3.8 | n.d.*) | 8.7 |
| 12 | n.d.*) | 1.7 | n.d.*) | 3.7 | n.d.*) | 8.3 |
| 18 | n.d.*) | 1.7 | n.d.*) | 3.7 | n.d.*) | 8.1 |

*) not determined

First, it can be seen from the particle size distribution that the liquid suspension prepared according to the present invention contains considerably smaller particles, although throughout the whole preparation process, no micro fluidization, in particular no micronization was performed.

Second, it can be seen that the liquid suspensions according to the present invention are characterized by an excellent long-term stability with respect to the particle size distribution since even after 18 months, the change in the d(0.1) value is less than 7%. As to the d(0.5) value, no change after 18 months can be observed, and during the observation period, the change is less than 3%. Also for the d(0.9) value, a very constant value is observed after 3, 6, 9, 12, and 18 months, and the change in d(0.9) after 18 months is less than 6%. In contrast thereto, the d(0.1), d(0.5) and d(0.9) values of the commercially available product were quite inconstant, even after only 3 months. In particular after 3 months, a considerable decrease in particle size was observed for the d(0.1) value (25%), and an increase in d(0.5) of more than 8%, and a high increase in d(0.9) of more than 14% were observed.

Again, it is shown that the pharmaceutical compositions of the present invention exhibit an excellent long-term stability with respect to the particle size distribution, in particular in combination with small particle sizes.

### 4. Sedimentation Test of the Liquid Suspension of the Present Invention

The liquid suspension of the present invention, prepared according to the process as described above, was filled in a vertically arranged glass cylinder and left at 60° C. for 6 weeks. After 6 weeks, essentially no phase separation was observed. This finding supports the results discussed above with respect to the particle size distribution since if phase separation and, thus, sedimentation had been observed, this would mean that a particle agglomeration and thus an increase in particle size would have taken place. The essential lack of phase separation therefore shows that the small particles of the inventive liquid suspension essentially keep their size.

### 5. Stability Test of Crystalline Form IV in Inventive Liquid Suspension

The liquid suspension of the present invention, prepared according to the process as described above, was subjected to storage conditions for 3 months, on the one hand at 25° C. and 60% relative humidity, on the other hand at 40° C. at 75% relative humidity (the latter being stress conditions). After the storage, the liquid suspension was subjected to XRD measurement in order to find out whether or not the initially pure crystalline form IV of posaconazole had changed, at least partially, its polymorphic structure. The resulting XRPD show that, after storage at the above-defined conditions and in particular after storage under stress conditions, only crystalline form IV of posaconazole is contained in the suspension. Therefore, posaconazole form IV shows polymorphic stability within the inventive formulation in the sense that no conversion to another polymorphic form was observed by measuring the XRPD.

The respective X-ray powder diffraction patterns (XRPD) were obtained with an X′Pert PRO diffractometer (PANalytical, Almelo, The Netherlands) equipped with a theta/theta coupled goniometer in transmission geometry, programmable XYZ stage with well plate holder, Cu—K alpha_{1,2} radiation source (wavelength 0.15419 nm) with a focussing mirror, a 0.5° divergence slit, a 0.02° soller slit collimator and a 0.5° anti-scattering slit on the incident beam side, a 2 mm anti-scattering slit, a 0.02° soller slit collimator, a Ni-filter and a solid state PIXcel detector on the diffracted beam side. The patterns were recorded at a tube voltage of 40 kV, tube current of 40 mA, applying a step size of 0.013° 2 theta with 80 s per step in the angular range of 2° to 40° 2 theta.

Therefore, it is shown that the inventive liquid suspension not only exhibits an advantageous long-term stability with respect to the particle size distribution, but also an advantageous long-term stability with respect to the stability of the polymorphic form, in particular polymorphic form IV of posaconazole, even under stress conditions.

### 6. Further Example for the Preparation of Crystalline Form IV of Posaconazole

a) Crude posaconazole was prepared according to the method disclosed in WO 2011/144653 A1, Example 5, on page 74, line 20, to page 76, line 14.

A suspension of 54.1 g crude posaconazole in 540 mL acetone and 160 mL water was heated to reflux, whereupon a clear solution was obtained. After filtration, the solution was allowed to stand overnight in a refrigerator at about 5° C., whereat crystallization was observed. The solid material was collected by filtration and dried under vacuum (<40 mbar) at room temperature overnight to obtain 47.3 g (87% yield) of posaconazole. The obtained posaconazole material mainly consisted of form II-S as described in WO 2011/003992 A1.
b) 20.3 g of the posaconazole material obtained from example 6a) were sieved (1 mm mesh size) and suspended together with 1.0 g posaconazole form IV seeds (obtained according to the process disclosed in example 2 of WO 2010/000668 A1) in 400 mL water and 100 mL methanol. The suspension was stirred overnight at 40° C. using a magnetic stirrer. The solid material was isolated by filtration, dried under vacuum at room temperature overnight and finally sieved (0.5 mm mesh size) to obtain 19.8 g (93% yield) of posaconazole form IV.
c) 20.0 g of the posaconazole material obtained from example 6a) were sieved (1 mm mesh size) and suspended together with 0.9 g posaconazole form IV seeds (obtained according to the process disclosed in example 2 of WO 2010/000668 A1) in 400 mL water and 100 mL methanol. The suspension was stirred overnight at 40° C. using a magnetic stirrer. The solid material was isolated by filtration, dried under vacuum at room temperature overnight and finally sieved (0.5 mm mesh size) to obtain 20.0 g (96% yield) of posaconazole form IV.
d) Parts of the posaconazole materials obtained from examples 6b) and 6c) were carefully mixed to obtain 25.6 g of posaconazole form IV.

### 7. Preparation of a Pharmaceutical Composition in the Form of a Liquid Suspension with Posaconazole Crystalline Form IV (not According to the Invention)

A liquid suspension was prepared having the following composition:

| Ingredient | Quantity/(g/5 mL) |
|---|---|
| Posaconazole Crystalline Form IV | 0.20000 |
| obtained according to Example 6 above | |
| Polysorbate 80 | 0.05000 |
| Simethicone | 0.01500 |
| Sodium Benzoate | 0.01000 |
| Sodium Citrate Dihydrate | 0.00300 |
| Citric Acid Monohydrate | 0.00750 |
| Glycerol | 0.50000 |
| Xanthan Gum | 0.01500 |
| Corn Syrup Solids | 1.44628 |
| Titanium Dioxide | 0.02000 |
| Cherry Flavor | 0.02000 |
| Purified Water | 3.40822 |
| Total Mass: | 5.69500 g |

The polysorbate 80 (NF quality) and the simethicone (NF quality) were mixed with purified water (USP quality) and homogenized with Ultra Turrax (60 seconds; 24,000 r.p.m.). Subsequently, posaconazole form IV prepared as described in Example 6 was suspended into this mixture and homogenized using an Ultra Turrax (90 seconds; 24,000 r.p.m.). Thereafter, sodium benzoate, sodium citrate dihydrate, citric acid monohydrate, glycerol, corn syrup solids and titanium dioxide (all NF quality) were admixed using the Ultra Turrax (120 seconds; 24,000 r.p.m.). Then, xanthan gum was added to the mixture followed by an overnight phase of hydratization without mixing. Afterwards the suspension was again homogenized using Ultra Turrax (120 seconds; 24,000 r.p.m.). Subsequently, cherry flavor was added.

### 8. Determination of the Particle Size Distribution: Comparison Between the Liquid Suspensions Containing Posaconazole Obtained According to Example 2 and Example 7: y

In the following table, the composition of the suspension obtained according to Example 7 and the composition of the liquid suspension prepared according to Example 2 are shown:

| | According to | According to |
|---|---|---|
| | Example 7** | Example 2* |
| Ingredient | mg/unit dose (5 mL) | mg/unit dose (5 mL) |
| Crystalline Posaconazole IV | 200.00 | 200.00 |
| Polysorbate 80 | 50.00 | 50.00 |
| Simethicone | 15.00 | 15.00 |
| Sodium Benzoate | 10.00 | 10.00 |
| Sodium Citrate Dihydrate | 3.00 | 3.00 |
| Citric Acid Monohydrate | 7.50 | 15.00 |
| Glycerol | 500.00 | 500.00 |
| Xanthan Gum | 15.00 | 5.00 |
| Liquid Glucose/Corn Syrup | 1446.28 | 1382.50 |
| Solids | | |
| Titanium Dioxide | 20.00 | 20.00 |
| Polyoxyl 40 Hydrogenated | — | 870.00 |
| Castor Oil | | |
| Cherry Flavor | 20.00 | 40.00 |

*according to the invention
**not according to the invention

The particle size distribution (Malvern) was determined as described in Example 3.1. The samples were stored at 25° C. at a relative humidity of 60% for 3 months and the respective particle sizes were measured at the starting point (that is after 0 months of storage) and after 3 months. The respective measurements were conducted at room temperature.

Surprisingly it has been observed that the liquid suspension obtained according to Example 2 exhibited an even higher stability when compared with the liquid suspension prepared according to Example 7. Unexpectedly, thus the increase in particle size after 3 month was even smaller for the composition according to Example 2 when compared with the composition according to Example 7 with a change in the d(0.9) value after 3 months of less than 4.9%. In contrast, the liquid suspension obtained according to Example 7 exhibited a change in the d(0.9) value of 20.9%.

Again, it has thus been shown that the pharmaceutical composition of the present invention exhibits an excellent long-term stability with respect to the particle size distribution.

### LITERATURE CITED

WO 02/080678 A1
WO 2010/000668 A1
WO 2011/144653 A1
U.S. Pat. No. 6,958,337 B2

1. A pharmaceutical composition, comprising crystalline posaconazole and one or more non-ionic surfactants, wherein at least one non-ionic surfactant is an ethoxylated hydrogenated castor oil.

2. The pharmaceutical composition of claim 1, wherein at least 90 weight-%, preferably at least 95 weight-%, more preferably at least 98 weight-% of the posaconazole comprised in the pharmaceutical composition are present as crystalline form IV, having an X-ray powder diffraction pattern comprising peaks at 2-theta angles of about 3.2°±0.2°, 6.6°±0.2°, 10.9°±0.2°, 16.9°±0.2°, 18.4°±0.2° and 25.1°±0.2°, measured with Cu—K alpha_{1,2} radiation, and/or having an attenuated total reflectance infrared spectrum comprising absorption bands at wavenumbers of about 3647 cm⁻¹±2 cm⁻¹, 3472 cm⁻¹±2 cm⁻¹, 2867 cm⁻¹±2 cm⁻¹, 1687 cm⁻¹±2 cm⁻¹, 1512 cm⁻¹±2 cm⁻¹, 1230 cm⁻¹±2 cm⁻¹, 1136 cm⁻¹±2 cm⁻¹, 916 cm⁻¹±2 cm⁻¹, 853 cm⁻¹±2 cm⁻¹, 819 cm⁻¹±2 cm⁻¹ and 681 cm⁻¹±2 cm⁻¹.

3. The pharmaceutical composition of claim 1 or 2, being a liquid dosage form, preferably an oral liquid dosage form, more preferably a liquid suspension.

4. The pharmaceutical composition of any of claims 1 to 3, wherein the at least one ethoxylated hydrogenated castor oil is selected from the group consisting of PEG-5 hydrogenated castor oil; PEG-7 hydrogenated castor oil; PEG-16 hydrogenated castor oil; PEG-20 hydrogenated castor oil; PEG-25 hydrogenated castor oil; PEG-30 hydrogenated castor oil; PEG-35 hydrogenated castor oil; PEG-40 hydrogenated castor oil; PEG-45 hydrogenated castor oil; PEG-50 hydrogenated castor oil; PEG-54 hydrogenated castor oil; PEG-55 hydrogenated castor oil; PEG-60 hydrogenated castor oil; PEG-80 hydrogenated castor oil; PEG-100 hydrogenated castor oil; PEG-200 hydrogenated castor oil, and a mixture of two or more of these hydrogenated castor oils, the at least one ethoxylated hydrogenated castor oil preferably being PEG-40 hydrogenated castor oil.

5. The pharmaceutical composition of any of claims 1 to 4, wherein the ratio of the weight of the at least one ethoxylated hydrogenated castor oil relative to the weight of posaconazole is in the range of from 1.5:1 to 8.5:1, preferably from 2.3:1 to 7.2:1, more preferably from 3.6:1 to 5.1:1, more preferably from 4.2:1 to 4.5:1.

6. The pharmaceutical composition of any of claims 1 to 5, additionally comprising at least one further non-ionic surfactant selected from the group consisting of polyoxyethylene derivatives of sorbitan esters of saturated C₁₀ to C₂₀ acids, polyoxyethylene derivatives of sorbitan esters of unsaturated C₁₀ to C₂₀ acids, and mixtures of two or more thereof.

7. The pharmaceutical composition of claim 6, wherein the ratio of the weight of the at least one further non-ionic surfactant relative to the weight of posaconazole is in the range of from 0.05:1 to 1:1, preferably from 0.1:1 to 0.5:1, more preferably from 0.2:1 to 0.3:1, more preferably from 0.22:1 to 0.28:1.

8. The pharmaceutical composition of any of claims 1 to 7, additionally comprising at least one buffering agent and/or at least one flavoring agent and/or at least one thickening agent, preferably at least one buffering agent and at least one flavoring agent and at least one thickening agent,
wherein the ratio of the weight of the at least one buffering agent relative to the weight of posaconazole is preferably in the range of from 0.05:1 to 0.2:1, more preferably from 0.07:1 to 0.15:1, more preferably from 0.08:1 to 0.1:1, the at least one buffering agent preferably being a mixture of sodium citrate dihydrate and citric acid monohydrate;
wherein the ratio of the weight of the at least one flavoring agent relative to the weight of posaconazole is preferably in the range of from 0.15:1 to 0.5:1, more preferably from 0.16:1 to 0.3:1, more preferably from 0.17:1 to 0.2:1, the at least one flavoring agent preferably being cherry flavor; and
wherein the ratio of the weight of the at least one thickening agent relative to the weight of posaconazole is preferably in the range of from 5:1 to 8.5:1, more preferably from 6:1 to 7.5:1, more preferably from 6.5:1 to 7:1, the at least one thickening agent preferably being a polysaccharide, more preferably being selected from the group consisting of glucose, xanthan gum, and a mixture thereof.

9. The pharmaceutical composition of any of claims 1 to 8, additionally comprising water, wherein the ratio of the weight of the water relative to the weight of posaconazole is preferably in the range of from 10:1 to 20:1, more preferably from 12:1 to 15:1, more preferably from 13:1 to 14:1.

10. The pharmaceutical composition of any of claims 1 to 9, having a particle size distribution characterized by a d(0.1) value in the range of from 1 to 3, preferably from 1 to 2 micrometer, a d(0.5) value in the range of from 3 to 5, preferably from 3 to 4 micrometer, and a d(0.9) value in the range of from 8 to 11, preferably from 8 to 9 micrometer.

11. The pharmaceutical composition of claim 10, having a long-term stability with regard to the particle size distribution of at least 6 months, preferably of at least 12 months, more preferably of at least 18 months, more preferably of at least 24 months, more preferably at least 36 months, wherein the long-term stability with regard to the particle size distribution is characterized in a change in the d(0.1) value of at most 10%, preferably of at most 7%, in a change in the d(0.5) value of at most 10%, preferably of at most 5%, and in a change in the d(0.9) value of at most 15%, preferably of at most 12%.

12. A process for the preparation of a pharmaceutical composition, preferably of the pharmaceutical composition according to any of claims 1 to 11, the process comprising
(aa) providing crystalline posaconazole, wherein preferably at least 90 weight-%, more preferably at least 95 weight-%, more preferably at least 98 weight-% of the posaconazole are present as crystalline form IV having an X-ray powder diffraction pattern comprising peaks at 2-theta angles of about 3.2°±0.2°, 6.6°±0.2°, 10.9°±0.2°, 16.9°±0.2°, 18.4°±0.2° and 25.1°±0.2°, measured with Cu—K alpha_{1,2} radiation, and/or having an attenuated total reflectance infrared spectrum comprising absorption bands at wavenumbers of about 3647 cm⁻¹±2 cm⁻¹, 3472 cm⁻¹±2 cm⁻¹, 2867 cm⁻¹±2 cm⁻¹, 1687 cm⁻¹±2 cm⁻¹, 1512 cm⁻¹±2 cm⁻¹, 1230 cm⁻¹±2 cm⁻¹, 1136 cm⁻¹±2 cm⁻¹, 916 cm⁻¹±2 cm⁻¹, 853 cm⁻¹±2 cm⁻¹, 819 cm⁻¹±2 cm⁻¹ and 681 cm⁻¹±2 cm⁻¹;
(bb) mixing one or more non-ionic surfactants with the posaconazole provided in (aa), wherein at least one non-ionic surfactant is an ethoxylated hydrogenated castor oil, the at least one ethoxylated hydrogenated castor oil preferably being selected from the group consisting of PEG-5 hydrogenated castor oil; PEG-7 hydrogenated castor oil; PEG-16 hydrogenated castor oil; PEG-20 hydrogenated castor oil; PEG-25 hydrogenated castor oil; PEG-30 hydrogenated castor oil; PEG-35 hydrogenated castor oil; PEG-40 hydrogenated castor oil; PEG-45 hydrogenated castor oil; PEG-50 hydrogenated castor oil; PEG-54 hydrogenated castor oil; PEG-55 hydrogenated castor oil; PEG-60 hydrogenated castor oil; PEG-80 hydrogenated castor oil; PEG-100 hydrogenated castor oil; PEG-200 hydrogenated castor oil, and a mixture of two or more of these hydrogenated castor oils, the at least one ethoxylated hydrogenated castor oil more preferably being PEG-40 hydrogenated castor oil;
wherein the at least one ethoxylated hydrogenated castor oil is admixed by a method comprising at least one sequence of homogenizing and mixing.

13. The process of claim 12, wherein during the entire process for the preparation of the pharmaceutical composition including the providing in (aa), no microfluidization, preferably no micronization is carried out.

14. The pharmaceutical composition according to any of claims 1 to 11 or the pharmaceutical composition obtainable or obtained by the process according claim 12 or 13 for use in a method of treating or preventing fungal infections in mammals in need of such treating or preventing such infections.

15. Use of a combination of crystalline posaconazole, preferably at least 90 weight-%, preferably at least 95 weight-%, more preferably at least 98 weight-% thereof being present as crystalline form IV, having an X-ray powder diffraction pattern comprising peaks at 2-theta angles of about 3.2°±0.2°, 6.6°±0.2°, 10.9°±0.2°, 16.9°±0.2°, 18.4°±0.2° and 25.1°±0.2°, measured with Cu—K alpha_{1,2} radiation, and/or having an attenuated total reflectance infrared spectrum comprising absorption bands at wavenumbers of about 3647 cm⁻¹±2 cm⁻¹, 3472 cm⁻¹±2 cm⁻¹, 2867 cm⁻¹±2 cm⁻¹, 1687 cm⁻¹±2 cm⁻¹, 1512 cm⁻¹±2 cm⁻¹, 1230 cm⁻¹±2 cm⁻¹, 1136 cm⁻¹±2 cm⁻¹, 916 cm⁻¹±2 cm⁻¹, 853 cm⁻¹±2 cm⁻¹, 819 cm⁻¹±2 cm⁻¹ and 681 cm⁻¹±2 cm⁻¹; and at least one ethoxylated hydrogenated castor oil for improving the long-term stability of liquid dosage forms comprising posaconazole with regard to the particle size distribution.
